(19) United States
(12) Patent Application Publication
(10) Pub. No.: US 2004/0181208 A1
(43) Pub. Date: Sep. 16, 2004

(54) Catheter reinforced with high yield strength wire

(76) Inventor: Matthew S. Poole, Bradford, MA (US)

Correspondence Address: MEDTRONIC VASCULAR, INC., IP LEGAL DEPARTMENT, 3576 UNOCAL PLACE, SANTA ROSA, CA, 95403, US

(21) Appl. No.: 10/389,397

(22) Filed: Mar. 14, 2003

(51) Int. Cl. A61M 25/00

(52) U.S. Cl. 604/527

(57) ABSTRACT

A tubular medical catheter having a filamentous reinforcement layer embedded between an inner liner and an outer shell is provided herein. The reinforcing layer includes at least one wire having high yield strength.

### FIELD OF THE INVENTION

The present invention relates to a catheter reinforced with high yield strength wire. More specifically, the present invention relates to a catheter having a braid comprising cobalt wire with a high modulus of elasticity.

### BACKGROUND OF THE INVENTION

A number of intravascular procedures are currently utilized to treat a stenosis within a body vessel of a human being. A common intravascular procedure is referred to as percutaneous transluminal coronary angioplasty (PTCA or hereinafter “angioplasty”). During a typical angioplasty procedure, a guidewire is initially positioned within the body vessel and a guiding catheter is positioned over the guidewire. Next, a balloon catheter having an inflatable balloon is advanced through the guiding catheter and vessel until the balloon is adjacent to the stenosis. Subsequently, inflation of the balloon compresses the stenosis and dilates the body vessel.

During many diagnostic or interventional catheterization procedures, it is necessary to route the catheter from an entry point, such as the femoral artery, to a target location within the vasculature. When properly placing a catheter into position, the catheter should be able to be turned, pulled, and pushed so that the distal end of the catheter can navigate the twists and turns of the blood vessels on its path to the final location. This requires that the catheter be rigid enough to transfer the torque being applied by the operator of the catheter, but also flexible enough so that the catheter will not damage any of the blood vessels of the patient.

One problem that has arisen in many catheters is that the catheter can be too stiff, which can prevent the catheter from passing through tortuous blood vessels. Alternately, the catheter can be too soft, which can result in the occurrence of kinks along the length of the catheter. In either of these situations, the usefulness of the catheter in the patient is limited.

A desired feature of many catheters is thin walls. One beneficial effect of a thin-walled catheter is that the thinner walls allow the lumen of the catheter to be larger which, in turn, allows a more diverse set of instruments, fluids, or drugs to be delivered to the location of interest. Alternatively, the lumen can remain the same size and the overall diameter of the catheter can be decreased, thus allowing delivery of the smaller catheter with an accompanying smaller puncture site in the individual being catheterized. A catheter having thinner walls, however, is more prone to kinking, which is undesirable. To help prevent or reduce kinking, catheters have been constructed to include support members, such as a braid encapsulated between an inner tube and an outer tube. U.S. Pat. No. 5,954,651, for example, reports a catheter having a braid wire with a high ultimate tensile strength. The rigidity and flexibility of the catheter, however, as well as the thickness of the tube wall, are still restricted by the materials used to construct the braid.

Accordingly, there is a need for a thin-walled medical catheter having decreased incidence of kinking while maintaining the strength, dimensions and other physical properties of existing catheters. The present invention addresses these needs, as well as other problems associated with existing medical catheters. The present invention also offers further advantages over the prior art and solves other problems associated therewith.

### SUMMARY OF THE INVENTION

The present invention is directed to medical catheters adapted for use within a body vessel. The medical catheter comprises a tubular catheter shaft having a distal end that fits within the body vessel. The tubular catheter shaft comprises an inner liner and outer shell. The medical catheter also comprises a reinforcing braid that is embedded between the inner liner and outer shell. The reinforcing braid has at least one wire that has a yield strength that is at least 10%, at least 20%, at least 30%, or at least 40% greater than the yield strength of stainless steel wire. The wire comprises an alloy material. In one embodiment, the wire comprises cobalt. In another embodiment, the wire comprises cobalt, chromium and nickel. In another embodiment, the wire comprises carbon, manganese, silicon, phosphorous, sulphur, chromium, nickel, molybdenum, cobalt, titanium, iron, and boron.

In some embodiments, the wire comprises about 0.005% to about 0.075% carbon by weight. In some embodiments, the wire comprises about 0.01% manganese to about 0.45% manganese by weight. In some embodiments, the wire comprises about 0.001% to about 0.03% sulphur by weight. In some embodiments, the wire comprises about 0.001% to about 0.045% phosphorous by weight. In some embodiments, the wire comprises about 10% to about 30% chromium by weight. In some embodiments, the wire comprises about 25% to about 45% nickel by weight. In some embodiments, the wire comprises about 4% to about 15% molybdenum by weight. In some embodiments, the wire comprises about 0.1% to about 5% titanium by weight. In some embodiments, the wire comprises about 0.1% to about 5% iron by weight. In some embodiments, the wire comprises about 0.001% to about 0.045% boron by weight. In some embodiments, the wire comprises about 0.01% to about 0.45% silicon by weight. In some embodiments, the wire comprises about 20% to about 45% cobalt by weight.

In other embodiments, the wire comprises about 0.005% to about 0.075% carbon by weight, about 0.01% manganese to about 0.45% manganese by weight, about 0.001% to about 0.03% sulphur by weight, about 0.001% to about 0.045% phosphorous by weight, about 10% to about 30% chromium by weight, about 25% to about 45% nickel by weight, about 4% to about 15% molybdenum by weight, about 0.1% to about 5% titanium by weight, about 0.1% to about 5% iron by weight, about 0.001% to about 0.045% boron by weight, about 0.01% to about 0.45% silicon by weight, and about 20% to about 45% cobalt by weight.

In other embodiments, the wire comprises about 0.015% to about 0.05% carbon by weight, about 0.05% manganese to about 0.3% manganese by weight, about 0.005% to about 0.02% sulphur by weight, about 0.005% to about 0.03% phosphorous by weight, about 15% to about 25% chromium by weight, about 30% to about 40% nickel by weight, about 7% to about 12% molybdenum by weight, about 0.5% to about 2% titanium by weight, about 0.5% to about 2% iron by weight, about 0.005% to about 0.03% boron by weight, about 0.05% to about 0.3% silicon by weight, and about 25% to about 40% cobalt by weight.

In some embodiments, the wire comprises MP35N, which is a precipitation hardenable cobalt metal-based alloy available from Fort Wayne Research Products Corp. (Fort Wayne, Ind.).

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a perspective view, in partial cutaway, of a medical catheter having features of the present invention;

FIG. 2 is an enlarged cutaway view of a portion of the medical catheter of FIG. 1;

FIG. 3 is a perspective illustration of the medical catheter positioned within a patient; and

FIG. 4 is a side view of a portion of the catheter shaft illustrating a groove.

### DETAILED DESCRIPTION OF THE INVENTION

The present invention is directed to a wire-reinforced catheter wherein the reinforcement, such as a braid, includes at least one high yield strength wire. Any medical catheter can be modified to comprise such a braid. Thus, the catheters described herein are merely exemplary; the invention should not be construed to be limited to only the catheters described herein.

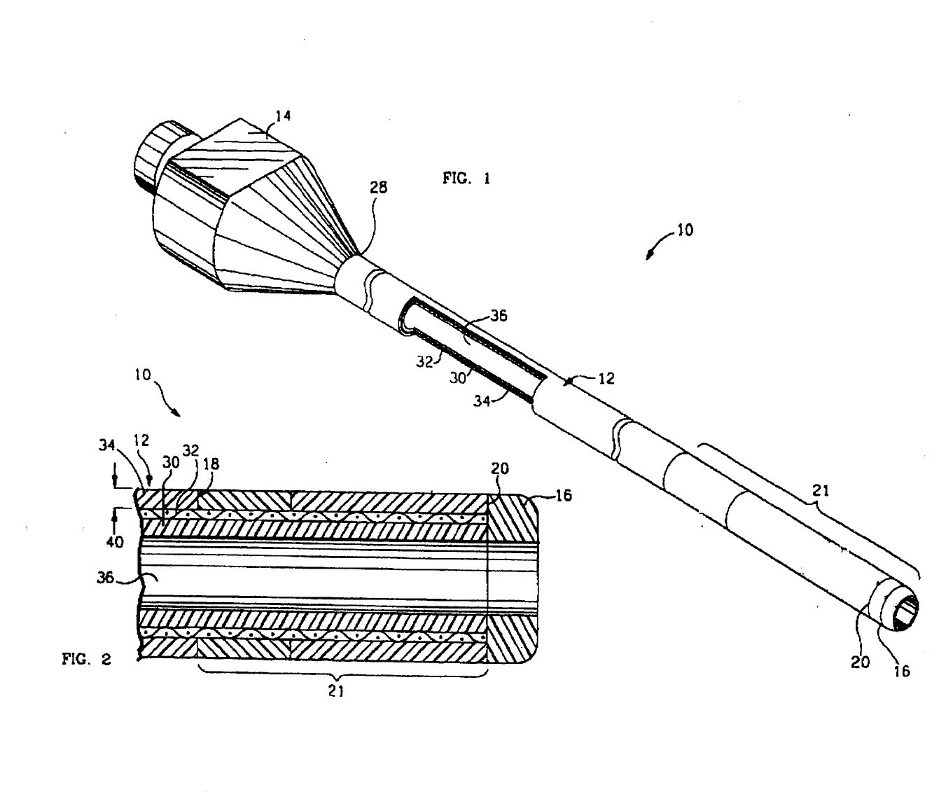

Referring to FIGS. 1, 2, and 4, a first embodiment of a medical catheter 10 having features of the present invention includes a tubular catheter shaft 12, a hub 14, and a tubular flexible tip 16. The hub 14 is secured to a proximal end 28 of the catheter shaft 12 while the flexible tip 16 is secured to the distal end 20 of the catheter shaft 12. The hub 14 and proximal end 28 are manipulated by the physician to position the medical catheter 10 in the body vessel 24. The flexible tip 16 assists in guiding the medical catheter 10 in the body vessel 24 and minimizes the trauma to the vessel 24, such as a coronary ostium (not shown).

The flexible tip 16 is made of a relatively soft material when compared to the catheter shaft 12. Suitable materials for the flexible tip 16 may include polymers, such as a polyether block amide (“PEBA”), having a hardness of about 40 D. Depending upon the materials utilized, the hub 14 and the flexible tip 16 can be thermally bonded or attached with an adhesive (not shown) to the catheter shaft 12. Those skilled in the art will recognize alternate ways to attach the hub 14 and flexible tip 16 and that alternate materials can be utilized for the flexible tip 16.

In the embodiment illustrated in FIGS. 1 and 2, the tubular catheter shaft 12 includes an inner liner 30, a reinforcing section 32, and an outer shell 34. The inner liner 30 is tubular and defines a lumen 36, which is sized and shaped to receive a guidewire 22 and/or different types of catheters (not shown). Typically, the inner liner 30 is manufactured by extruding a polymer such as PEBA or nylon, which provides good flexibility and movement over the guidewire 22. A suitable inner liner 30 has an inner diameter of between about 0.08 and 0.09 inches and an inner liner thickness of about 1.5 mils. A coating (not shown) may be added to the lumen 36 of the inner liner 30 to facilitate movement of the guidewire 22 and the balloon catheter within the lumen 36.

The outer shell 34 provides support to the catheter shaft 12 and covers the reinforcing section 32 to protect the body vessel 24 from the reinforcing section 32. Further, the outer shell 34 prevents the reinforcing section 32 from unwrapping. The outer shell 34 is tubular and coaxial with the inner liner 30 and the reinforcing section 32. A suitable outer shell 34 has an inner diameter of about 0.1 inches and a shell thickness 40 of about 2.5 mils.

Typically, the outer shell 34 is manufactured by extruding a polymer over the reinforcing section 32. A suitable shell material for the outer shell 34 is a nylon sold under the trademark “TROGAMID” by Creanova (Somerset, N.J.). The shell material may have a hardness of approximately 81 Shore D. Optionally, a lubricious coating (not shown) may be added to the outer shell 34 to facilitate movement of the catheter shaft 12 within the vessel 24.

The reinforcing section 32 enhances the torsional strength and prevents or reduces kinking of the catheter shaft 12 during movement of the medical catheter 10 in the body vessel 24. The reinforcing section 32 is positioned between the inner liner 30 and the outer shell 34 and is substantially coaxial with the inner liner 30 and the outer shell 34. The reinforcing section 32 may be formed by braiding wire mesh around the inner liner 30. A braid pattern may be selected from a variety of braid types, such as one-over-one, two-over-two, one-over two, etc. Alternatively, reinforcing section 32 may be formed without inter-weaving the filaments, as by laying one helical wire layer over another helical wire layer having an opposite direction or “hand.” Subsequently, the outer shell 34 is formed around the reinforcing section 32 by applying materials making up the outer shell.

The reinforcing section 32 can be continuous along the catheter shaft 12 and the transition region 21, or can be present as several discontinuous sections along the catheter shaft 12 and the transition region 21. As a result thereof, the medical catheter 10 provided herein has improved tracking and torsional characteristics within the vessel and the medical catheter 10 is relatively easy to manipulate by the physician.

In some embodiments, the reinforcing section 32 may be a braid that has at least one wire that has a yield strength that is at least 10%, at least 20%, at least 30%, or at least 40% greater than the yield strength of stainless steel wire. The yield strengths of the wires of the present invention can be compared to the yield strength of stainless steel wire, for example, by comparing the yield strength of each at 90% cold work (hardening). The ultimate tensile strength of the wires of the present invention may be similar to that of stainless steel wire, despite the higher yield strength of the wires of the present invention.

A suitable wire may comprise about 0.005% to about 0.075% carbon by weight. In some embodiments, the wire comprises about 0.01% to about 0.45% manganese by weight. In some embodiments, the wire comprises about 0.001% to about 0.03% sulphur by weight. In some embodiments, the wire comprises about 0.001% to about 0.045% phosphorous by weight. In some embodiments, the wire comprises about 10% to about 30% chromium by weight. In other embodiments, the wire comprises about 25% to about 45% nickel by weight. In some embodiments, the wire comprises about 4% to about 15% molybdenum by weight. In some embodiments, the wire comprises about 0.1% to about 5% titanium by weight. In some embodiments, the wire comprises about 0.1% to about 5% iron by weight. In some embodiments, the wire comprises about 0.001% to about 0.045% boron by weight. In some embodiments, the wire comprises about 0.01% to about 0.45% silicon by weight. In some other embodiments, the wire comprises about 20% to about 45% cobalt by weight. As used throughout the present description, the term “about” means ±5% of the value being modified (e.g., about 100 means 95 to 105). The braid can be in a wire form, such as in a mesh, or in another form as desired by one skilled in the art.

In other embodiments, the wire comprises about 0.005% to about 0.075% carbon by weight, about 0.01% manganese to about 0.45% manganese by weight, about 0.001% to about 0.03% sulphur by weight, about 0.001% to about 0.045% phosphorous by weight, about 10% to about 30% chromium by weight, about 25% to about 45% nickel by weight, about 4% to about 15% molybdenum by weight, about 0.1% to about 5% titanium by weight, about 0.1% to about 5% iron by weight, about 0.001% to about 0.045% boron by weight, about 0.01% to about 0.45% silicon by weight, and about 20% to about 45% cobalt by weight.

In other embodiments, the wire comprises about 0.015% to about 0.05% carbon by weight, about 0.05% manganese to about 0.3% manganese by weight, about 0.005% to about 0.02% sulphur by weight, about 0.005% to about 0.03% phosphorous by weight, about 15% to about 25% chromium by weight, about 30% to about 40% nickel by weight, about 7% to about 12% molybdenum by weight, about 0.5% to about 2% titanium by weight, about 0.5% to about 2% iron by weight, about 0.005% to about 0.03% boron by weight, about 0.05% to about 0.3% silicon by weight, and about 25% to about 40% cobalt by weight. In some embodiments, the wire comprises MP35N.

The braid can be fabricated from round or profile wire stock. Typical round wire diameters are about 0.0005 inch to about 0.005 inch, with profile wire sizes varying from a width to height ratio of 1:1 to 8:1, having a minimum height of 0.0005 inches to a maximum width of 0.005 inches. The braid pattern, defined in pics per inch (ppi), may vary depending on the desired pushability. In regions where pushability is required, the pattern may be in the range from about 10 to about 40 ppi while in the regions where flexibility and kink resistance are essential, the pattern may be in the range of from about 50 to about 150 ppi. In addition, changing the ppi, adjusting the diameter of the wire, and/or changing the width to height ratio may further alter the pushability characteristics of the catheter. In addition, the ppi may be changed from region to region within the same catheter.

A reinforcing wire comprising carbon, manganese, silicon, phosphorous, sulphur, chromium, nickel, molybdenum, cobalt, titanium, iron, and boron, such as MP35N, rather than a reinforcing wire comprising stainless steel, provides the catheter with increased resistance to kinking because of the increase in the wire's yield strength. Such a catheter can retain desirably thin walls, yet be more resistant to kinking. Further, MP35N has a higher modulus of elasticity when compared to stainless steel (34,000,000 psi for MP35N as compared to 26,000,000-28,000,000 psi for type 304 stainless steel, which is commonly used in catheters). Using a wire comprising MP35N also provides for more responsive handling of the entire catheter. The high modulus of elasticity allows the user to place more stress on the catheter without causing it to exceed the yield strength of the reinforcing wire, which can lead to kinking, either in torsion or in bending. Another benefit of MP35N is that the metal's inherent increase in the modulus of elasticity allows the thin-walled catheter to resist kinking failurs of other thin-walled catheters (e.g., in bending, torsion, or crushing, etc.) as compared to a catheter of the same dimensions and having stainless steel reinforcing wire that has comparable dimensions and degree of cold working (see, Table 1 below). In addition, such benefits of the MP35N material are not specific to wire having flat or round profiles

TABLE 1

| | |
|---|---|
| | Yield Strength |
| % Cold Work | (KPSI) |

| (Hardening) | Stainless Steel 304 V | MP35N |
|---|---|---|
| 75 | 200 | 329 |
| 80 | 215 | 332 |
| 90 | 245 | 345 |
| 93 | 250 | 346 |
| 95 | 280 | 362 |

Those skilled in the art will recognize alternate ways to manufacture the inner liner 30, the reinforcing section 32, and the outer shell 34, and that alternate materials can be utilized for the inner liner 30, the reinforcing section 32, and the outer shell 34. Those skilled in the art will also recognize alternate ways to apply the reinforcing section 32 on the inner liner 30. Examples of other reinforcing sections 32 that are applied in a particular design can be found in U.S. Pat. No. 5,954,651, which is incorporated herein by reference in its entirety.

the medical catheter 10 illustrated herein is utilized to guide smaller catheters (not shown) and is commonly referred to as a guiding catheter. FIG. 3 illustrates a portion of the medical catheter 10 and a guidewire 22 positioned in a body vessel 24 of a patient 26 during a procedure. The location of entry into the patient 26 and the location of the distal end 20 in the patient 26 are merely exemplary.

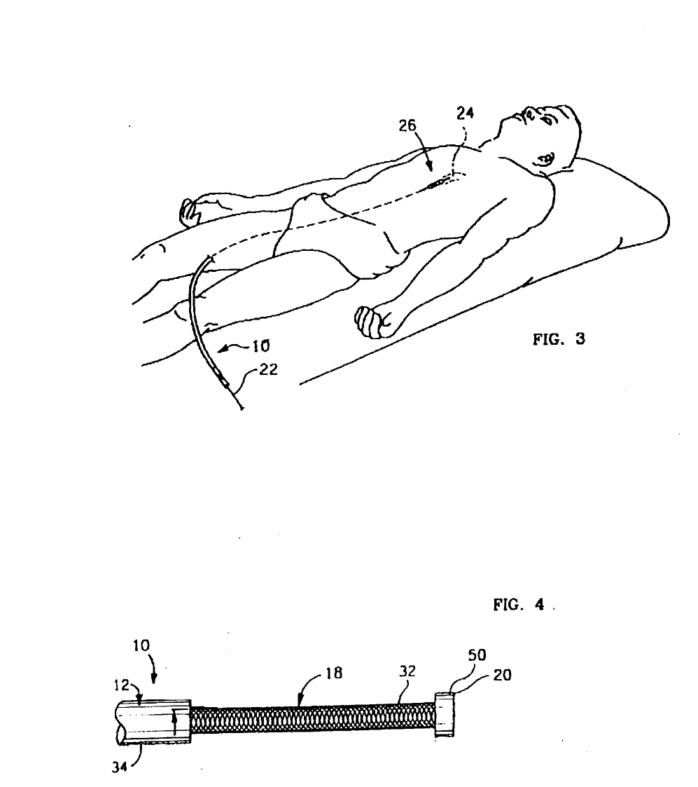

As illustrated in FIG. 4, the catheter shaft 12 can optionally include a groove 18, which is cut out of the catheter shaft 12 near a distal end 20 of the catheter shaft 12, as described in U.S. Pat. No. 6,059,769, which is incorporated herein by reference in its entirety. In some embodiments, the tubular catheter shaft 12 may also comprise unfilled or low-loaded inner liner 30 and/or outer shell 34. For example, the inner liner 30 and outer shell 34 each, independently, may include a radiopaque material and/or filler and/or colorant, such that the total content of the radiopaque material and/or filler and/or colorant in inner liner 30 and/or outer shell 34 is between about 0.1% and about 10%, or between about 0.1% and about 5%, or between about 0.1% and about 2% of the total weight making up the inner liner 30 and/or outer shell 34. In some embodiments, the inner liner 30 and outer shell 34 each, independently, may exclude a radiopaque material and/or filler and/or colorant, thus having 0% by weight of the total weight making up the inner liner 30 and/or outer shell 34. An unfilled inner liner 30 and/or outer shell 34 have the advantages of retaining mechanical integrity and modulus of elasticity.

While the particular medical catheter 10 as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

What is claimed is:

1. A medical catheter adapted for use within a body vessel, the medical catheter comprising:
a tubular catheter shaft comprising an inner liner and an outer shell and having a distal end which fits within the body vessel; and
a reinforcing layer comprising at least one wire embedded between the inner liner and outer shell;
wherein the wire has a yield strength that is at least 10% greater than the yield strength of a stainless steel wire having comparable degree of cold work.

2. The medical catheter of claim 1 wherein the reinforcing layer is a braid.

3. The medical catheter of claim 1 wherein the wire has a yield strength that is at least 20% greater than the yield strength of a stainless steel wire having comparable degree of cold work.

4. The medical catheter of claim 1 wherein the wire has a yield strength that is at least 30% greater than the yield strength of a stainless steel wire having comparable degree of cold work.

5. The medical catheter of claim 1 wherein the wire has a yield strength that is at least 40% greater than the yield strength of a stainless steel wire having comparable degree of cold work.

6. The medical catheter of claim 1 wherein the wire comprises cobalt.

7. The medical catheter of claim 6 wherein the wire comprises about 20% to about 40% cobalt by weight.

8. The medical catheter of claim 6 wherein the wire further comprises chromium and nickel.

9. The medical catheter of claim 8 wherein the wire comprises about 10% to about 30% chromium by weight.

10. The medical catheter of claim 8 wherein the wire comprises about 25% to about 40% nickel by weight.

11. The medical catheter of claim 8 wherein the wire further comprises carbon, manganese, silicon, phosphorus, sulphur, molybdenum, titanium, iron, and boron.

12. The medical catheter of claim 11 wherein the wire comprises about 0.005% to about 0.075% carbon by weight.

13. The medical catheter of claim 11 wherein the wire comprises about 0.01% to about 0.45% manganese by weight.

14. The medical catheter of claim 11 wherein the wire comprises about 0.001% to about 0.03% sulphur by weight.

15. The medical catheter of claim 11 wherein the wire comprises about 0.001% to about 0.045% phosphorous by weight.

16. The medical catheter of claim 11 wherein the wire comprises about 4% to about 15% molybdenum by weight.

17. The medical catheter of claim 11 wherein the wire comprises about 0.1% to about 5% titanium by weight.

18. The medical catheter of claim 11 wherein the wire comprises about 0.1% to about 5% iron by weight.

19. The medical catheter of claim 11 wherein the wire comprises about 0.001% to about 0.045% boron by weight.

20. The medical catheter of claim 11 wherein the wire comprises about 0.01% to about 0.45% silicon by weight.

21. The medical catheter of claim 11 wherein the wire comprises about 0.005% to about 0.075% carbon by weight, about 0.01% to about 0.45% manganese by weight, about 0.001% to about 0.03% sulphur by weight, 0.001% to about 0.045% phosphorous by weight, about 10% to about 30% chromium by weight, about 25% to about 45% nickel by weight, about 4% to about 15% molybdenum by weight, about 0.1% to about 5% titanium by weight, about 0.1% to about 5% iron by weight, about 0.001% to about 0.045% boron by weight, about 0.01% to about 0.45% silicon by weight, and about 20% to about 45% cobalt by weight.

22. The medical catheter of claim 11 wherein the wire comprises about 0.015% to about 0.05% carbon by weight, about 0.05% to about 0.3% manganese by weight, about 0.005% to about 0.02% sulphur by weight, about 0.005% to about 0.03% phosphorous by weight, about 15% to about 25% chromium by weight, about 30% to about 40% nickel by weight, about 7% to about 12% molybdenum by weight, about 0.5% to about 2.0% titanium by weight, about 0.5% to about 2.0% iron by weight, about 0.005% to about 0.03% boron by weight, about 0.05% to about 0.3% silicon by weight, and about 25% to about 40% cobalt by weight.

23. The medical catheter of claim 11 wherein the wire comprises about 0.025% carbon by weight, about 0.15% manganese by weight, about 0.01% sulphur by weight, about 0.015% phosphorous by weight, about 20% chromium by weight, about 35% nickel by weight, about 10% molybdenum by weight, about 1.0% titanium by weight, about 1.0% iron by weight, about 0.015% boron by weight, about 0.15% silicon by weight, and about 35% cobalt by weight.

24. The medical catheter of claim 11 wherein the wire comprises MP35N.
